(19) United States
(12) Patent Application Publication
(10) Pub. No.: US 2002/0045831 A1
(43) Pub. Date: Apr. 18, 2002

(54) Scanner for a medical optical imaging device employing suppression of optical reflections

(76) Inventors: Robert H. Wake, Cooper City, FL (US); Richard J. Grable, Plantation, FL (US); William Dieckmann, Miami Springs, FL (US)

Correspondence Address: SHLESINGER, ARKWRIGHT & GARVEY LLP, PATENT, TRADEMARK & COPYRIGHT LAW, 3000 SOUTH EADS STREET, ARLINGTON, VA, 22202, US

(21) Appl. No.: 09/829,443

(22) Filed: Apr. 10, 2001

Related U.S. Application Data
(60) Provisional application No. 60/197,415, filed on Apr. 14, 2000.

(51) Int. Cl. A61B 6/00

(52) U.S. Cl. 600/473; 600/476; 356/435

(57) ABSTRACT

A scanner for a medical optical imaging device, comprises an illumination source positioned to direct emitted light into a breast positioned below a support surface; a plurality of detectors positioned to detect light emerging from the breast; and a container disposed below the illumination source and the detectors adapted to trap light reflected from the breast.

### RELATED APPLICATION

This a nonprovisional application claiming the priority benefit of provisional application Ser. No. 60/197,415, filed Apr. 14, 2000, which is hereby incorporated by reference.

### FIELD OF THE INVENTION

The present invention generally relates to diagnostic medical imaging apparatus and more particularly to a mammography machine that employs a near-infrared laser as a radiation source.

### BACKGROUND OF THE INVENTION

Cancer of the breast is a major cause of death among the American female population. Effective treatment of this disease is most readily accomplished following early detection of malignant tumors. Major efforts are presently underway to provide mass screening of the population for symptoms of breast tumors. Such screening efforts will require sophisticated, automated equipment to reliably accomplish the detection process.

The x-ray absorption density resolution of present photographic x-ray methods is insufficient to provide reliable early detection of malignant tumors. Research has indicated that the probability of metastasis increases sharply for breast tumors over 1 cm size. Tumors of this size rarely produce sufficient contrast in a mammogram to be detectable. To produce detectable contrast in photographic mammograms, 2-3 cm dimensions are required. Calcium deposits used for inferential detection of tumors in conventional mammography also appear to be associated with tumors of large size. For these reasons, photographic mammography has been relatively ineffective in the detection of this condition.

Most mammographic apparatus in use today in clinics and hospitals require breast compression techniques which are uncomfortable at best and in many cases painful to the patient. In addition, x-rays constitute ionizing radiation which injects a further risk factor into the use of mammographic techniques as most universally employed.

Ultrasound has also been suggested, as in U.S. Pat. No. 4,075,883, which requires that the breast be immersed in a fluid-filled scanning chamber. U.S. Pat. No. 3,973,126 also requires that the breast be immersed in a fluid-filled chamber for an x-ray scanning technique.

In recent times, the use of light and more specifically laser light to noninvasively peer inside the body to reveal the interior structure has been investigated. This technique is called optical imaging. Optical imaging and spectroscopy are key components of optical tomography. Rapid progress over the past decade have brought optical tomography to the brink of clinical usefulness. Optical wavelength photons do not penetrate in vivo tissue in a straight line as do x-ray photons. This phenomenon causes the light photons to scatter inside the tissue before the photons emerge out of the scanned sample.

Because x-ray photon propagation is essentially straight-line, relatively straight forward techniques based on the Radon transform have been devised to produce computed tomography images through use of computer algorithms. Multiple measurements are made through 360° around the scanned object. These measurements, known as projections, are used to back project the data to create an image representative of the interior of the scanned object.

The detectable signals in an optical breast scanning device are at a very low level. Ambient light must be excluded from the scanning area. Reflections inside the scanner can cause image artifacts or otherwise cause the reconstructed images to be of little use.

### OBJECTS AND SUMMARY OF THE INVENTION

It is an object of the present invention to provide a scanner for a medical optical imaging device that suppresses optical reflections within the scanning chamber to minimize formation of artifacts to the reconstructed image.

It is another object of the present invention to provide a scanner for a medical optical imaging device that excludes ambient light from entering the scanning chamber.

It is another object of the present invention to provide a scanner for a medical optical imaging device that suppresses reflections inside the optical cavity between the breast and the photodetector.

In summary, the present invention provides a scanner for a medical optical imaging device, comprising an illumination source positioned to direct emitted light into a breast positioned below a support surface; a plurality of detectors positioned to detect light emerging from the breast; and a container disposed below the illumination source and the detectors adapted to trap light reflected from the breast.

The present invention also provides a scanner for a medical optical imaging device, comprising an illumination source positioned to direct emitted light into a breast positioned below a support surface; a plurality of detectors positioned to detect light emerging from the breast; and a collimator having a plurality of holes associated with the respective plurality of detectors to restrict the field of view of the detectors. The holes include non-smooth inside surfaces.

The present invention further provides a scanner for a medical optical imaging device, comprising a scanning chamber including an illumination source positioned to direct emitted light into a breast and a plurality of detectors positioned to detect light emerging from the breast. The scanning chamber includes inside surfaces coated with low-reflectivity material.

The present invention further provides a scanner for a medical optical imaging device, comprising a scanning chamber including an illumination source positioned to direct emitted light into a breast and a plurality of detectors positioned to detect light emerging from the breast. The scanning chamber includes slanted vertical surfaces to direct light from a horizontal plane.

These and other objects of the present invention will become apparent from the following detailed description.

### BRIEF DESCRIPTIONS OF THE DRAWINGS

FIG. 1 is a schematic side elevational view of a medical optical imaging device showing a patient positioned on a support with her breast pendent within a scanning chamber made in accordance with the present invention,.

FIG. 2 is a schematic cross-sectional view of a container disposed below a scanner to capture reflected light from the breast during scanning.

FIG. 3 is a top plan view taken along line 3-3 of FIG. 2, illustrating a honeycomb structure used as a light trap.

FIG. 4 is an enlarged partial cross-sectional view of a side wall of the container shown in FIG. 2, showing the relationship between the slant angle and the plane of the data acquisition.

FIG. 5 is a perspective view of a collimator made in accordance with the present invention, showing a plurality of openings to restrict the field of view of detectors.

FIG. 6 is schematic plan view of the scanner, showing the relationship between the patient's breast, illumination beam, collimator, detector field of view, and the detector.

FIG. 7 is an enlarged schematic cross-sectional view through line 7-7 of FIG. 5, showing a light trap for minimizing off-axis light from reaching the detector.

### DETAILED DESCRIPTION OF THE INVENTION

A medical optical imaging device is disclosed in U.S. Pat. Nos. 5,692,511, 6,100,520, 6,130,958, which are hereby incorporated by reference.

Referring to FIG. 1, a patient 2 is positioned prone on a scanning table 4 with one breast 6 pendulant in a scanning chamber 8. A medical optical imaging scanner 10 comprises a collimator 12 secured to an orbit plate 14 and an elevator plate 16. The collimator 12 is associated with detectors 13 (see FIG. 5). The orbit plate 14 is orbited through one circle around the breast to obtain one slice of data. The elevator plate 16 is moved vertically by drive screws 18 to position the orbit plate 14 at different vertical locations where the orbit plate 14 is again orbited through one circle around the breast to obtain another slice of data. A side curtain 20 is fixed to the underside of the table 4 and the elevator plate 16 to form a barrier for ambient light for the scanning chamber 8 defined by the side curtain 20, the orbit plate 14, the elevator plate 16 and a hollow container 21, such as a cylinder.

The side curtain 20 is foldable vertically to allow it to expand and retract as the vertical plate 16 is lowered or raised. The side curtain 20 includes slanted vertical surfaces 23. The side curtain 20 is advantageously made from low or nonreflective material.

Referring to FIG. 2, the hollow cylinder 21 has a vertical wall having an inside surface formed into a series of non-vertical steps 22 adapted to direct internal reflections, generally indicated at 24, downwardly towards the bottom and away from the collimator 12 and the detectors 13. The reflections 24 originate from the scanning beam 40 impinging on the breast 6. A bottom wall 26 of the hollow cylinder 21 is provided with a honeycomb structure 28 with openings 30 directed upwardly towards the breast. The honeycomb structure 28 advantageously traps any stray reflections within the hollow cylinder 21 and prevents the reflections from being directed back towards the breast and the collimator 12.

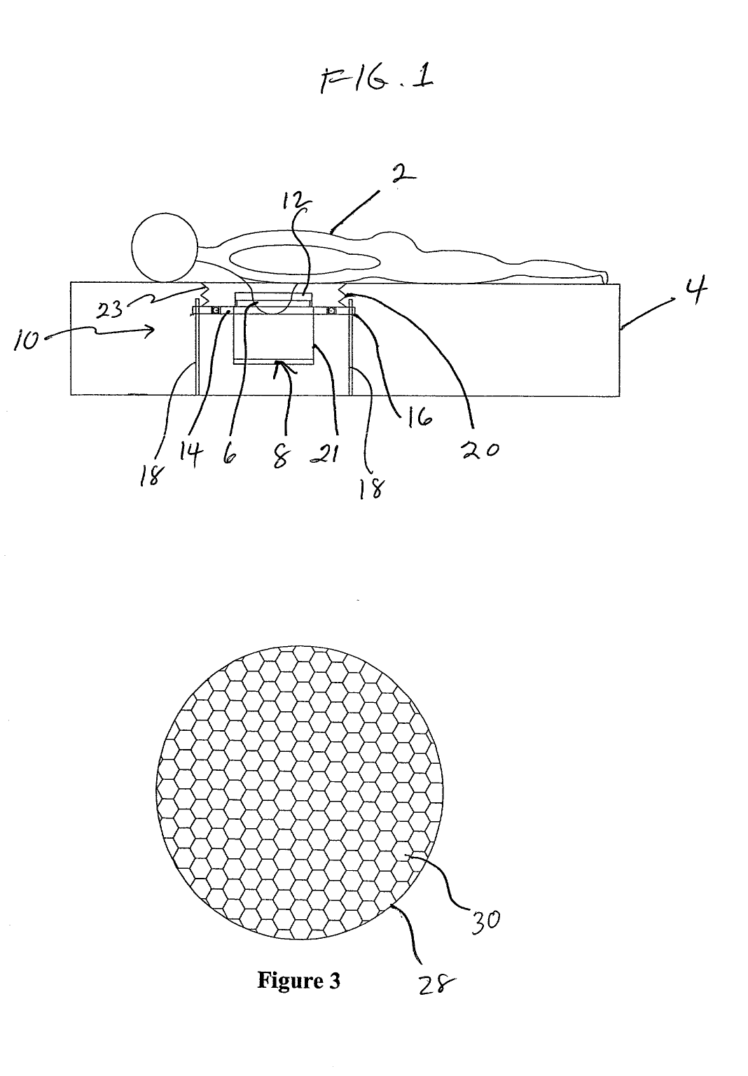

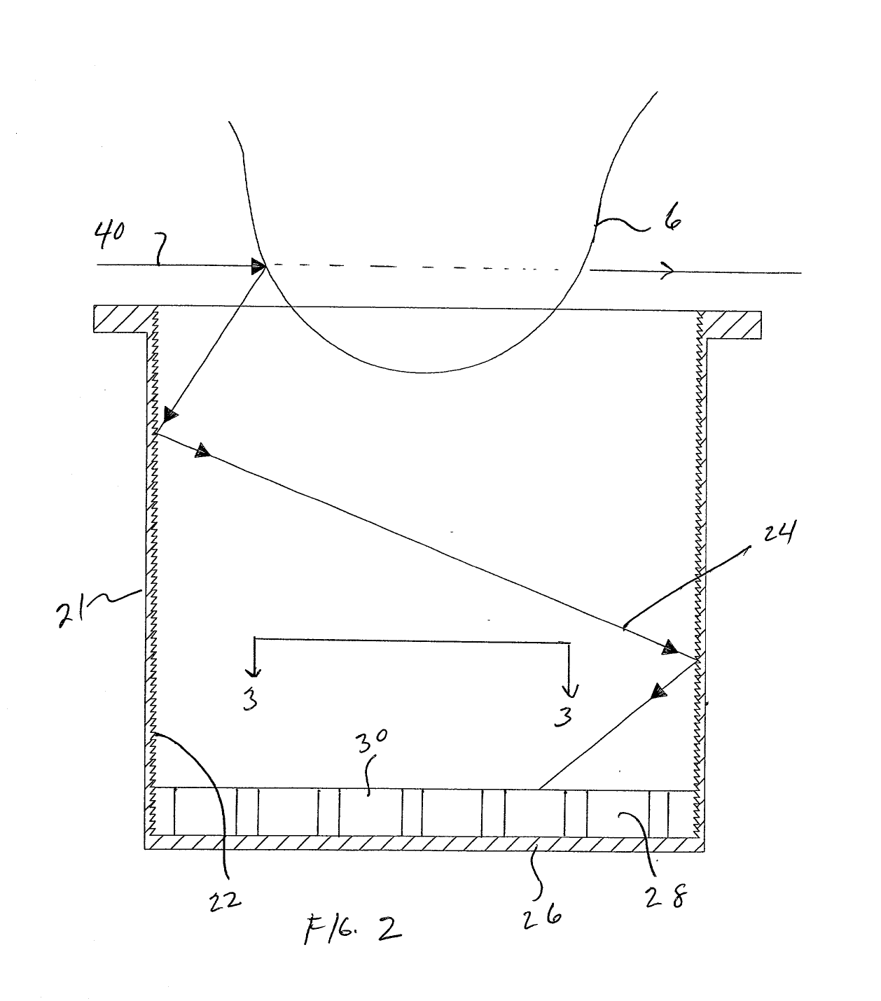

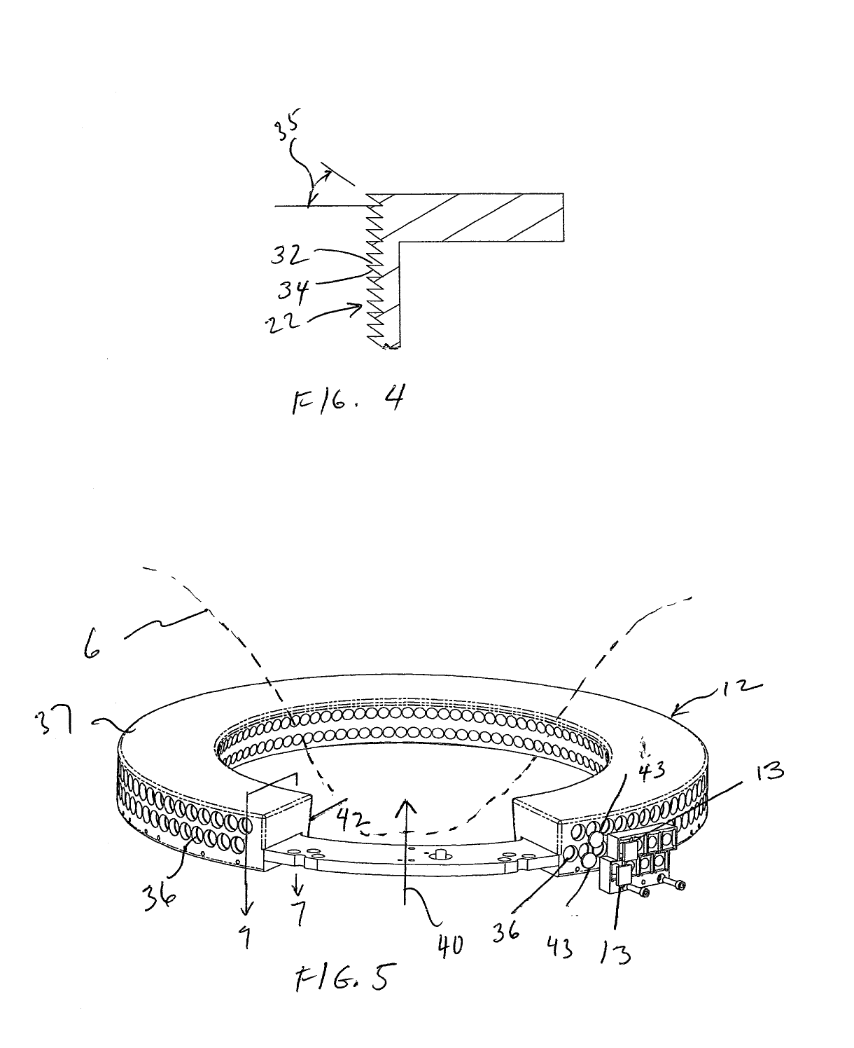

The steps 22 are preferably formed with horizontal portions 32 and inclined portions 34, as best shown in FIG. 4. The steps 22 are configured to direct reflected light away from the scan plane, generally indicated by the scanning beam 40 shown in FIG. 2. The angle 35 between the portions 32 and 34 is configured to cause downward reflections of the stray light. The inside surfaces of the hollow cylinder, including the steps and the honeycomb structure, are painted with flat-black paint to make the surfaces low or non-reflective. The openings of the honeycomb structure 28 are preferably hexagonal, as shown in FIG. 3; however, circular, square, triangular, pentagonal or other geometric shapes would also work.

Referring to FIG. 5, the collimator 12 comprises a series of holes 36 through a body 37 that arches around the breast 6. Detectors 13 are positioned at the end of each hole 36 to detect light coming from the breast 6 due to the laser beam 40 impinging on the breast during scanning. The collimator 12 has a vertical surface 42 that faces the breast. The surface 42 is preferably slanted at about 15° off the vertical to direct any stray reflections downwardly toward the hollow cylinder 21 and away from other openings 36. A lens 43 may be placed in front of each detector 13 to increase light collection capability.

Within the scanning chamber 8, any surfaces facing the breast is advantageously made low or nonreflective with flat black paint and are slanted from the vertical. In this manner, the chances of any stray reflection finding its way into the holes 36 of the collimator 12 are minimized.

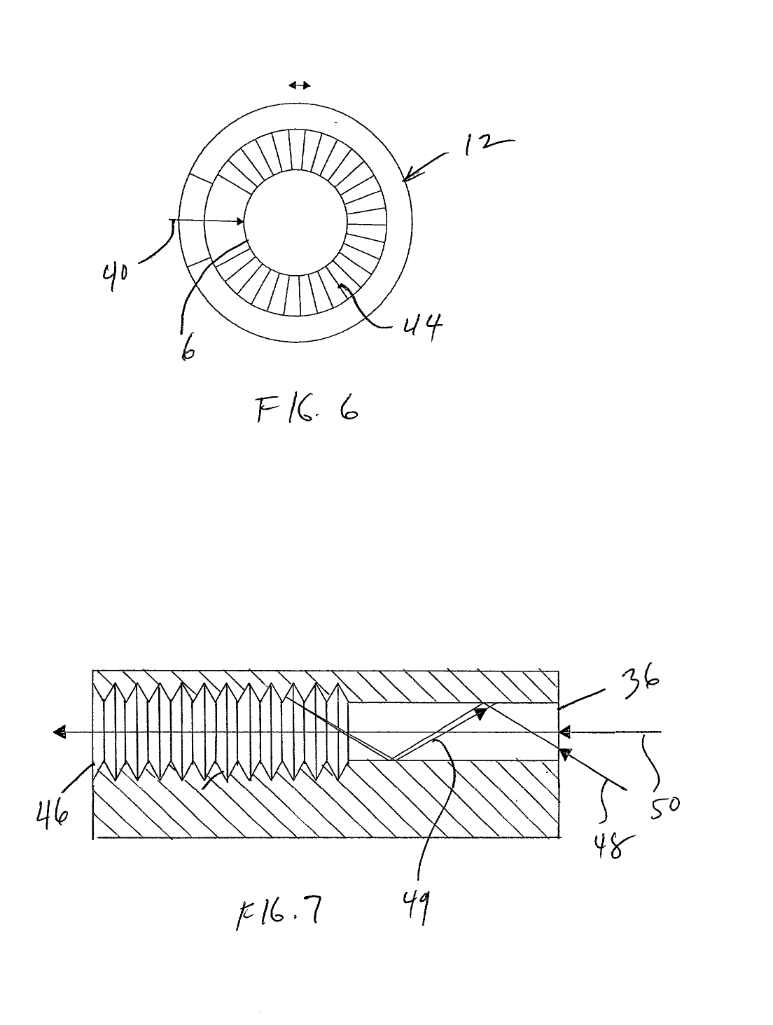

The collimator 12 is shown schematically in plan view in FIG. 6. Each opening 36 has a field of view, schematically indicated at 44 to restrict the amount and direction of light that can be detected by the detectors 13.

Referring to FIG. 7, a portion of the inside surface of each hole 36 is made non-smooth, such as by providing a series of grooves with slanted walls, or threading the opening with a fine pitch screw thread 46, to significantly reduce the occurrence of off-axis light, generally indicated at 48, from the reaching the detector 13 disposed at the other end of the hole. The side walls of the thread 46 change the reflection path of the light 48, as generally indicated at 49. The length of the openings 36 limits the field of view of the respective detector 13. Off-axis light 48 is generally reflected light which is not useful. Through axis light 50, which has passed through the breast, is used for image reconstruction.

While this invention has been described as having preferred design, it is understood that it is capable of further modification, uses and/or adaptations following in general the principle of the invention and including such departures from the present disclosure as come within known or customary practice in the art to which the invention pertains, and as may be applied to the essential features set forth, and fall within the scope of the invention or the limits of the appended claims.

We claim:

1. A scanner for a medical optical imaging device, comprising:
a) an illumination source positioned to direct emitted light into a breast positioned below a support surface;
b) a plurality of detectors positioned to detect light emerging from the breast; and
c) a container disposed below said illumination source and said detectors adapted to trap light reflected from the breast.

2. A scanner as in claim 1, wherein said container is cylindrical.

3. A scanner as in claim 1, wherein:
a) said container includes an inside vertical wall surface; and
b) said inside vertical wall surface includes angular steps.

4. A scanner as in claim 3, wherein said angular steps each includes a horizontal surface and an angular surface directed upwardly.

5. A scanner as in claim 3, wherein said vertical wall surface is coated with low reflectivity material.

6. A scanner as in claim 5, wherein said low reflectivity material is flat black paint.

7. A scanner as in claim 1, wherein said container includes an inside bottom surface.

8. A scanner as in claim 7, wherein said inside bottom surface includes vertically directed honeycomb structure with openings directed upwardly.

9. A scanner as in claim 8, wherein said openings are hexagonal.

10. A scanner as in claim 7, wherein said inside bottom surface is coated with low-reflectivity material.

11. A scanner as in claim 10, wherein said material is flat black paint.

12. A scanner as in claim 1, and further comprising a side curtain disposed around the breast to exclude ambient light from the breast.

13. A scanner as in claim 12, wherein said side curtain is foldable vertically.

14. A scanner as in claim 1, and further comprising:
a) a collimator to be disposed around the breast; and
b) said collimator including a slanted vertical surface facing the breast.

15. A scanner as in claim 14, wherein said slanted vertical surface is disposed toward said container at about 150 from a vertical reference line.

16. A scanner as in claim 14, wherein:
a) said collimator includes a plurality of openings; and
b) said openings include inside surfaces with grooves with slanted walls.

17. A scanner as in claim 16, wherein said grooves are made with screw threads.

18. A scanner for a medical optical imaging device, comprising:
a) an illumination source positioned to direct emitted light into a breast positioned below a support surface;
b) a plurality of detectors positioned to detect light emerging from the breast; and
c) a collimator having a plurality of holes associated with respective said plurality of detectors to restrict the field of view of said detectors, said holes including non-smooth inside surfaces.

19. A scanner as in claim 18, wherein said inside surfaces include grooves with slanted walls.

20. A scanner as in claim 18, wherein said inside surfaces are provided with screw threads.

21. A scanner as in claim 18, wherein said collimator includes a slanted vertical surface facing the breast.

22. A scanner as in claim 21, wherein said slanted vertical surface is disposed toward said container at about 150 from a vertical reference line.

23. A scanner for a medical optical imaging device, comprising:
a) a scanning chamber including an illumination source positioned to direct emitted light into a breast and a plurality of detectors positioned to detect light emerging from the breast; and
b) said scanning chamber including inside surfaces coated with low-reflectivity material.

24. A scanner as in claim 23, wherein said material is flat black paint.

25. A scanner for a medical optical imaging device, comprising:
a) a scanning chamber including an illumination source positioned to direct emitted light into a breast and a plurality of detectors positioned to detect light emerging from the breast; and
b) said scanning chamber including slanted vertical surfaces to direct light from a horizontal plane.

26. A scanner as in claim 25, wherein:
a) said chamber includes a container disposed below said illumination source and said detectors;
b) said container includes an inside vertical wall surface; and
c) said inside vertical wall surface includes angular steps.

27. A scanner as in claim 26, wherein said angular steps each includes a horizontal surface and an angular surface directed upwardly.

28. A scanner as in claim 26, wherein said container is cylindrical.

29. A scanner as in claim 25, wherein said chamber includes a side curtain disposed around the breast to exclude ambient light from the breast.

30. A scanner as in claim 29, wherein said side curtain is foldable vertically with slanted vertical surfaces.

31. A scanner for a medical optical imaging device, comprising:
a) an illumination source positioned to direct emitted light into a breast positioned below a support surface;
b) a plurality of detectors positioned to detect light emerging from the breast; and
c) a collimator having a plurality of holes associated with respective said plurality of detectors to restrict the field of view of said detectors, said holes including non-smooth inside surfaces.

32. A scanner as in claim 31, wherein said inside surfaces include grooves with slanted walls.

33. A scanner as in claim 31, wherein said inside surfaces are provided with screw threads.

34. A scanner as in claim 31, wherein said collimator includes a slanted vertical surface facing the breast.

35. A scanner as in claim 34, wherein said slanted vertical surface is disposed toward said container at about 150 from a vertical reference line.
